(19) United States
(12) Patent Application Publication
(10) Pub. No.: US 2026/0233024 A1
(43) Pub. Date: Aug. 13, 2026

(54) IRIDIUM COMPLEX, PHOTOSENSITIZER, AND COMPOSITION FOR PHOTODYNAMIC THERAPY

(71) Applicant: UNIST(ULSAN NATIONAL INSTITUTE OF SCIENCE AND TECHNOLOGY), Ulsan (KR)

(72) Inventors: Taehyuk KWON, Ulsan (KR); Chaegyu LEE, Ulsan (KR)

(73) Assignee: UNIST(ULSAN NATIONAL INSTITUTE OF SCIENCE AND TECHNOLOGY), Ulsan (KR)

(21) Appl. No.: 19/121,426

(22) PCT Filed: Oct. 11, 2023
(86) PCT No.: PCT/KR2023/015616

§ 371 Date: Apr. 13, 2026

(30) Foreign Application Priority Data
Oct. 17, 2022 (KR) 10-2022-0133353
Aug. 14, 2023 (KR) 10-2023-0106492

(51) Int. Cl. A61N 5/06; A61K 31/555; A61K 41/00; A61P 35/00; C07F 15/00

(52) U.S. Cl. CPC A61N 5/062; A61K 31/555; A61K 41/0057; A61P 35/00; C07F 15/0033; A61N 2005/0611

(57) ABSTRACT

The present invention relates to an iridium complex, a photosensitizer, and a composition for photodynamic therapy and to an iridium complex, a photosensitizer, and a composition for photodynamic therapy wherein the iridium complex is positively charged and when used, exhibits high intracellular accumulation rates and a high capacity for generating reactive oxygen species and not only demonstrates superior cancer cell killing capabilities compared to existing photosensitizers but also shows a synergistic effect with conventional iridium complexes.

### CROSS-REFERENCE TO PRIOR APPLICATIONS

This Application is a National Stage Patent Application of PCT International Application No. PCT/KR2023/015616 (filed on Oct. 11, 2023), which claims priority to Korean Patent Application Nos. 10-2022-0133353 (filed on Oct. 17, 2022) and 10-2023-0106492 (filed on Aug. 14, 2023), which are all hereby incorporated by reference in their entirety.

### BACKGROUND

The present disclosure relates to an iridium complex, a photosensitizer, and a composition for photodynamic therapy and, more specifically, to an iridium complex, a photosensitizer, and a composition for photodynamic therapy, exhibiting a high intracellular accumulation rate and a high reactive oxygen generation ability by utilizing a positively charged iridium complex while exhibiting a high cancer cell killing ability as well as a synergy effect with conventional iridium complexes compared to existing photosensitizers

A photodynamic therapy is a treatment method that minimizes the pain of cancer patients via photochemical reactions, in which light is irradiated on cancer tissues after administration of a photosensitizer into a patient to allow the photosensitizer to generate reactive oxygen species through a photochemical reaction, thereby killing cancer cells by oxidizing their biomolecules. In the case of this treatment method, owing to low physical burden on the patient, treatment can be resumed within 1 month, and it is also possible to use it in combination with existing cancer treatment methods.

However, ‘Photofrin®’ and ‘Radachlorin®’, which are photosensitizers generally used in the actual photodynamic therapy, have significantly low efficiency in producing reactive oxygen species in aqueous systems, such that very strong light energy (>200-300 J/cm²) must be applied for the photodynamic therapy.

Therefore, in order to minimize adverse effects caused by strong light energy and laser irradiation time, iridium complexes that exhibit very high efficiency in generation of reactive oxygen species in aqueous systems are being studied as the photosensitizer for the photodynamic therapy, but improvements are needed for the low cell killing efficiency and inherent toxicity of the iridium complex.

### SUMMARY

An object of the present disclosure is to provide an iridium complex, a photosensitizer, and a composition for photodynamic therapy using the same, which may increase an intracellular accumulation rate of a photosensitizer and enhance an efficiency of generating reactive oxygen by providing a positively charged iridium complex, thereby increasing a cell killing efficiency against cancer cells.

In addition, another object of the present disclosure is to provide an iridium complex, a photosensitizer, and a composition for photodynamic therapy using the same, which may reduce the physical burden on a patient during a photodynamic therapy by identifying a synergy effect with a known iridium complex.

To address technical objects above, one embodiment of the present disclosure provides an iridium complex represented by the following Chemical Formula 1:

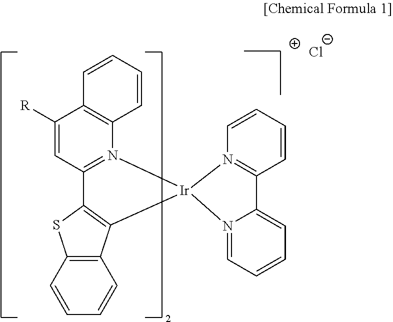

(where R is selected from a methyl group (CH₃) or a trifluoromethyl group (CF₃).)

A photosensitizer of the present disclosure is an iridium complex-based photosensitizer that has a high intracellular accumulation rate due to cationic properties and exhibits a high cell killing efficiency against various cancer cell types based on superior reactive oxygen generation efficiency, thereby improving an efficiency in a photodynamic therapy.

In addition, the iridium complex of the present disclosure may be applied to a treatment method that decreases an amount of photosensitizer during the photodynamic therapy to lead to reduction in the physical burden on a patient by identifying a synergy effect with a conventional iridium complex.

### BRIEF DESCRIPTION OF THE DRAWINGS

FIGS. 1A and 1B illustrate molecular structures of iridium complexes in Examples 1 and 2 (HIrb, FIrb) and Comparative Examples 1 to 3 (HIra, FIra, FIrp).

FIGS. 2A and 2B illustrates schematic diagrams of synergism among combinations of iridium complexes in Example 1 (HIrb), Comparative Example 4 (B2), and Comparative Example 5 (TIr3) and molecular structures of the iridium complexes.

FIG. 3A shows a graph illustrating changes in an intracellular accumulation rate depending on the charge of an iridium complex.

FIG. 3B shows images associated with merging of intracellular signals (green) and mitotracker signals (red) of an iridium complex in Example 1.

FIG. 4A shows a graph illustrating changes in absorption of 9,10-anthracenediyl-bis(methylene)dimalonic acid (ABDA) depending on an amount of singlet oxygen (¹O₂) generated over light irradiation time.

FIG. 4B shows a graph illustrating a fluorescence intensity which increases in response to type I reactive oxygen of Dihydrorhodamine 123 (DHR123) which is a fluorescent substance.

FIG. 4C shows images illustrating the intracellular fluorescence analysis of 2′,7′-dichlorodihydrofluorescein diacetate (DCFH-DA, an intracellular type I reactive oxygen marker) depending on the presence of light irradiation (0.25 sun, 25 mW/cm², 1 min).

FIGS. 5A-5E illustrates graphs illustrating a synergy effect with iridium complexes of Examples 1 and 2 and Comparative Examples 4 and 5.

FIGS. 6A-6F illustrates graphs illustrating toxicity of iridium complexes.

FIGS. 7A-7E illustrates schematic diagrams of micellized synergistic combination of TIr3 and B2 and graphs illustrating effects thereof.

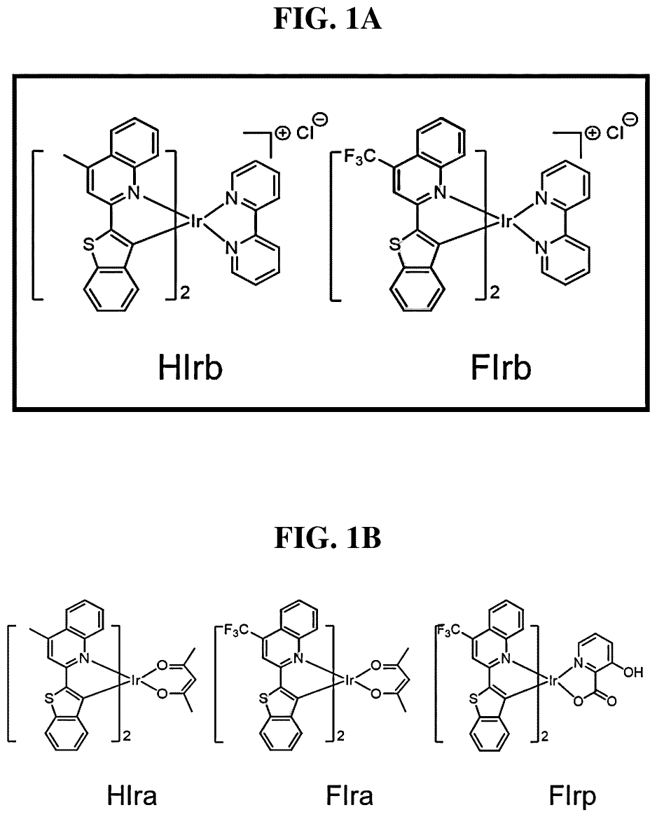

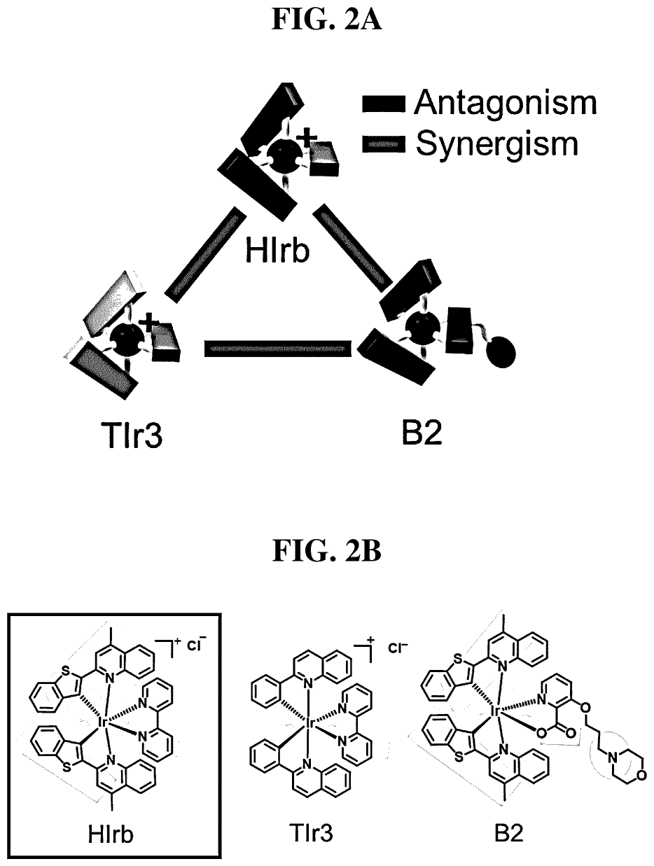

### DETAILED DESCRIPTION

Hereinafter, preferred embodiments of the present disclosure will be described in more detail with reference to the attached drawings. Prior to this, terms or words used in this specification and claims should not be interpreted as limited to conventional or dictionary meanings, but interpreted as meanings and concepts that conform to the technical idea of the present disclosure based on the principle that the inventor may appropriately define the concept of the term in order to explain one's own disclosure in the best way.

Therefore, embodiments described herein and configurations illustrated in the drawings are only the most preferred embodiments of the present disclosure and do not represent all the technical ideas of the present disclosure, such that it should be understood that there may be various equivalents and modified examples that may replace those at the time of filing this application.

The present disclosure relates to an iridium complex, a photosensitizer, and a composition for photodynamic therapy, wherein the iridium complex according to one embodiment of the present disclosure may ensure a high accumulation rate in cells, particularly within mitochondria, by having cationic properties and provide a high killing effect against various cancer cells based on high efficiency in generating reactive oxygen.

According to one embodiment of the present disclosure, the iridium complex may be a compound represented by the following Chemical Formula 1.

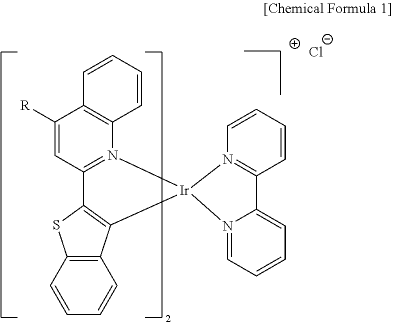

Wherein, R may be selected from a methyl group (CH₃) or a trifluoromethyl group (CF₃).

The iridium complex may be selected from the following Chemical Formula 2 (HIrb) or Chemical Formula 3 (FIrb).

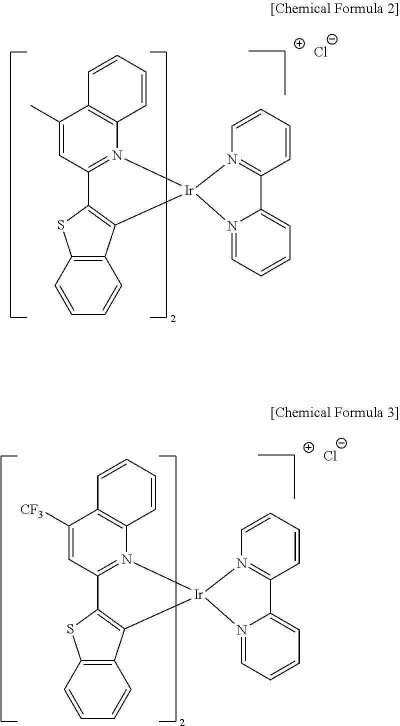

According to one embodiment of the present disclosure, the iridium complex may be a compound represented by the following Chemical Formula 7 or 9.

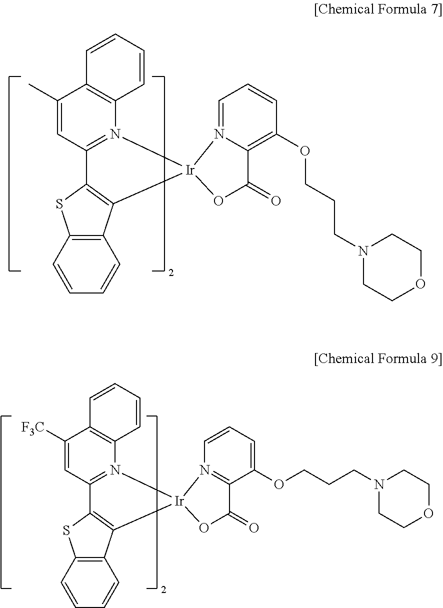

The iridium complex may form a positive charge on the iridium complex by adopting bipyridine as a sub-ligand, thereby enhancing the high intracellular accumulation rate, especially the accumulation rate of the iridium complex within mitochondria.

In addition, the present disclosure provides a photosensitizer including the iridium complex represented by Chemical Formula 1, wherein the photosensitizer may be positively charged.

In addition, the present disclosure relates to a composition for photodynamic therapy including the photosensitizer as an active ingredient, wherein, according to one embodiment of the present disclosure, the composition may include a photosensitizer including the iridium complex of the present disclosure and a carrier.

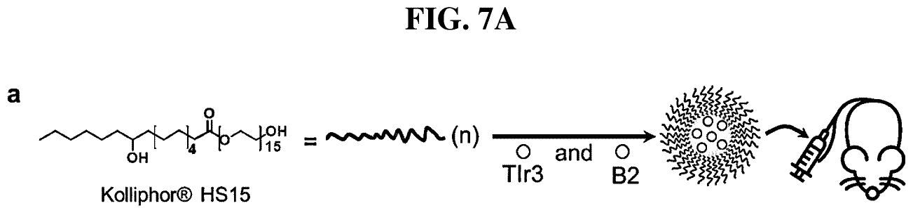

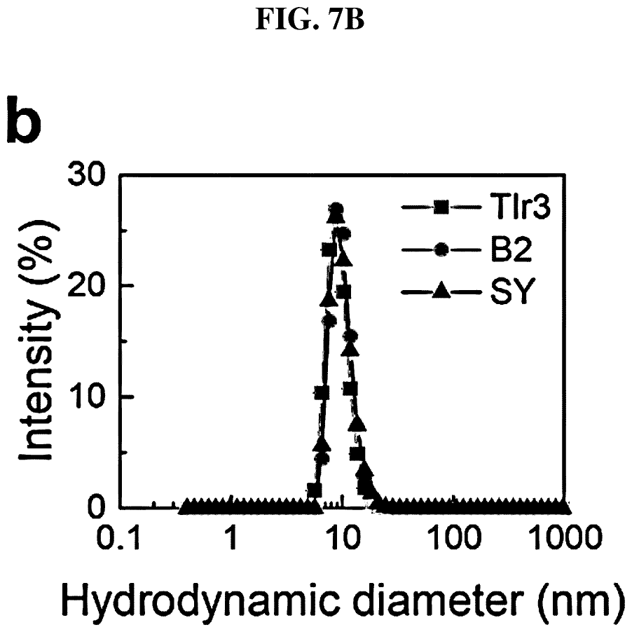

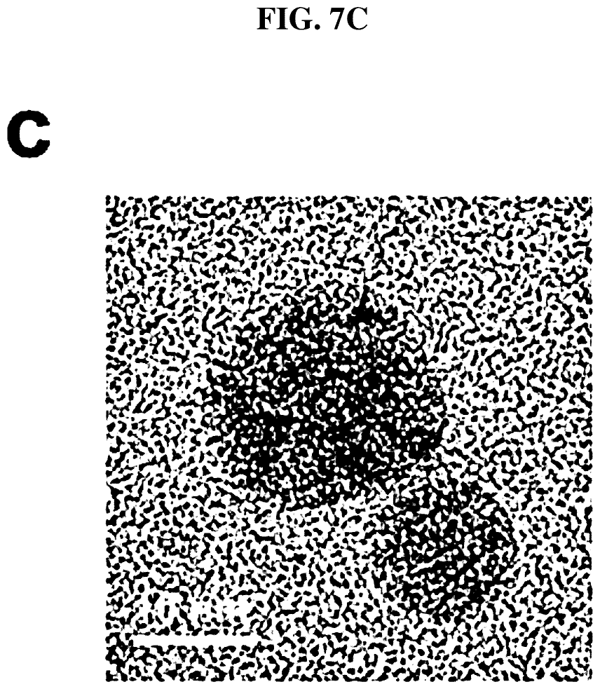

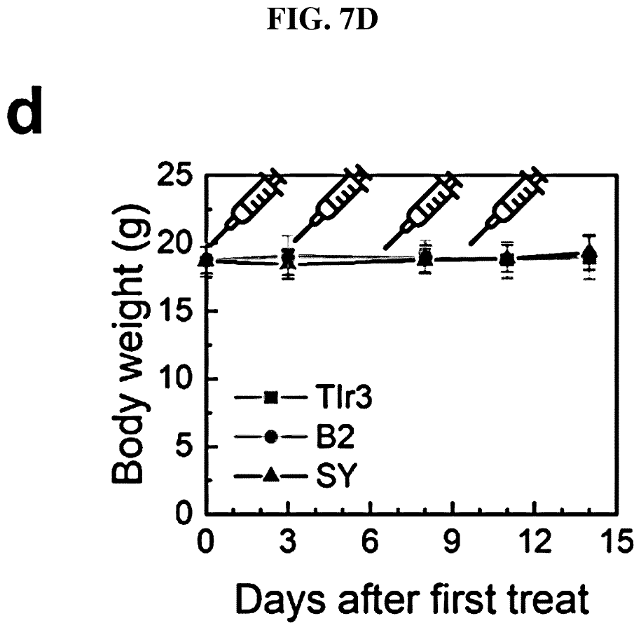

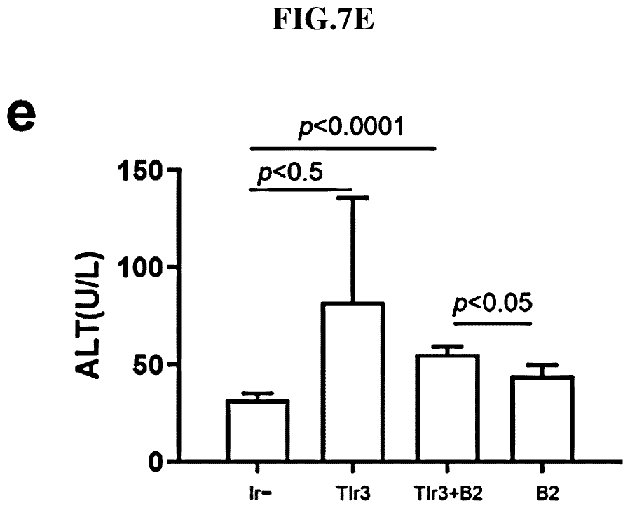

FIGS. 7A-7E illustrates schematic diagrams of micellized synergistic combination (SY) of TIr3 and B2 using Kolliphor HS 15 surfactant (FIG. 7A). As a result, when TIr3, B2, and 0.5 mg/ml TIr3+0.5 mg/ml B2 (SY) materials are dissolved in distilled water at a concentration of 1 mg/mL using 5% Kolliphor HS15, it was found via dynamic light scattering (DLS) that nanoparticles with a size of approximately 10 nm were formed (FIG. 7B). In addition, the size of the SY combination was reidentified through transmission electron microscopy (TEM) (FIG. 7C). When the micelle combination was injected into nude mice four times at a concentration of 10 mg/kg, no significant difference was observed in body weight, revealing that the drug combination had no effect on the activity of animals (FIG. 7D). After four administrations of the drug, the results of hepatotoxicity (alanine aminotransferase, ALT) on the 17^{th} day are shown in FIG. 7E.

The iridium complex is included in a pharmaceutically effective amount, and the pharmaceutically effective amount refers to an amount sufficient to achieve a therapeutic effect when the iridium complex according to the present disclosure is applied to the diagnosis, prevention, and treatment of a disease or illness.

The iridium complex is included as a pharmaceutically effective ingredient in the composition, and may be included, for example, in an amount of 0.001 parts by weight to less than 100 parts by weight, 0.001 parts by weight to 50 parts by weight or 0.1 parts by weight to 10 parts by weight, with respect to 100 parts by weight of the composition, but is not limited thereto.

It is preferable that the carrier is a pharmaceutically acceptable carrier. For example, it may include one or more selected from the group consisting of, but is not limited to, lactose, dextrose, sucrose, sorbitol, mannitol, starch, acacia gum, calcium phosphate, alginate, gelatin, calcium silicate, microcrystalline cellulose, cellulose, polyvinylpyrrolidone, water, syrup, methyl cellulose, methyl hydroxybenzoate, propyl hydroxybenzoate, talc, magnesium stearate, and mineral oil. In addition, the composition may further include, but is not limited to, one or more selected from the group consisting of a lubricant, humectant, sweetener, flavoring agent, emulsifier, suspending agent, and preservative.

The composition may be prepared in a unit dose form by being formulated using a pharmaceutically acceptable carrier and/or excipient according to a method that may be easily performed by a person skilled in the art to which the present disclosure pertains, or may be prepared by placing it in a multi-dose container. At this time, the formulation may be in the form of a solution, suspension, or emulsion in an oil or aqueous medium, and may further include a dispersant or stabilizer.

The composition may be administered orally or parenterally, for example, intravenously, intraperitoneally, intramuscularly, subcutaneously, or locally, and may also be administered rectally, by inhalation, or transdermally, but is not limited thereto.

The appropriate dosage of the composition varies depending on factors such as the formulation method, administration method, patient's age, weight, and sex, severity of disease symptoms, food, administration time, administration route, excretion rate, and response sensitivity, and a generally skilled physician may easily determine and prescribe an effective dosage for the desired treatment.

The composition may be applied to the photodynamic therapy to be utilized for cancer treatment using photochemical reaction. The cancer may be any one or more selected from the group consisting of cervical cancer, colon cancer, pancreatic cancer, lung cancer, breast cancer, stomach cancer, melanoma, skin cancer, bile duct cancer, neuroendocrine tumor, ovarian cancer, liver cancer, bronchial cancer, nasopharyngeal cancer, laryngeal cancer, bladder cancer, colorectal cancer, brain cancer, prostate cancer, bone cancer, head and neck cancer, thyroid cancer, parathyroid cancer, and ureteral cancer, but is not limited thereto.

In the present disclosure, through administering of a photosensitizer including the iridium complex of the present disclosure to a patient and then irradiating of light on cancer tissues, a photochemical reaction is induced by the photosensitizer, and reactive oxygen species generated through the reaction oxidize biomolecules of cancer cells to lead to death. In the irradiating of light, light energy of 0.001 J/cm² or greater, preferably 0.1 J/cm² or greater, more preferably 1 J/cm² to 300 J/cm² may be irradiated for 1 second or longer, preferably 10 seconds or longer, more preferably 1 second to 5 hours, but is not limited thereto.

The present disclosure relates to a photodynamic therapeutic method using the iridium complex according to the present disclosure, wherein, according to one embodiment of the present disclosure, the method includes administering the composition to a subject; allowing a predetermined time for the composition to accumulate within target cells of the subject; and irradiating light energy to the target cells of the subject.

The photodynamic therapeutic method may enable treatment of a cancer through a mechanism in which a photosensitizer is accumulated in target cells and light energy is irradiated to cause the photosensitizer to generate reactive oxygen species through a photochemical reaction, thereby killing cancer cells.

The administering is conducted in an oral or parenteral method, and such the administration methods are as mentioned above.

The subject may be a mammal, such as, but not limited to, a human, mouse, rat, guinea pig, dog, cat, horse, cow, pig, monkey, chimpanzee, bear, rabbit, baboon, or rhesus macaque.

The composition for photodynamic therapy of the present disclosure may further include one or more selected from the group consisting of B2 represented by Chemical Formula 7, TIr3 represented by Chemical Formula 8, and B4 represented by Chemical Formula 9.

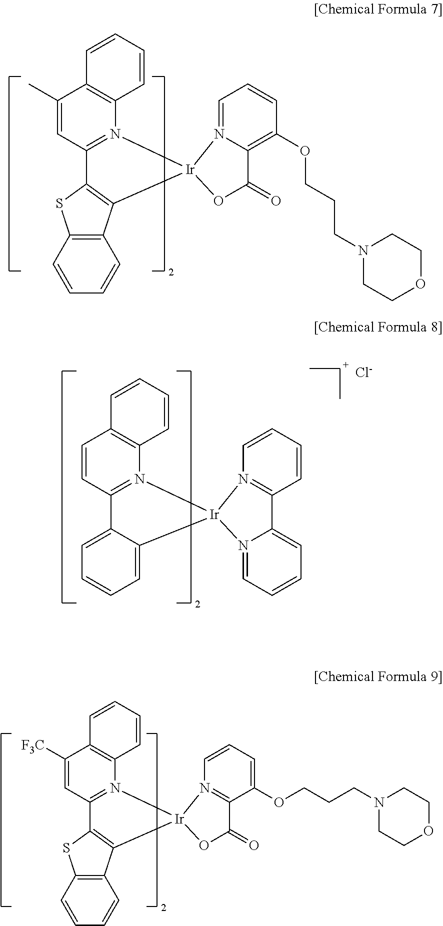

The iridium complex of the present disclosure exhibits a synergy effect in the photodynamic therapy through combination with the above B2, B4 and TIr3, thereby reducing the amount of toxic photosensitizer required and thus drastically lowering the burden on patients who receive treatment.

At this time, the cancer to be treated upon co-administration of the iridium complex of the present disclosure and B2, B4, or TIr3 is preferably a cervical cancer, but is not limited thereto.

In addition, the composition of the present disclosure may accumulate inside mitochondria by electrostatic attraction depending on the characteristics of the iridium complex having a positive charge after being administered to a patient and enable cancer treatment by generating reactive oxygen inside mitochondria upon irradiation with light energy.

Hereinafter, the present disclosure will be described in more detail through examples. These examples are merely intended to illustrate the present disclosure, and it will be apparent to those skilled in the art that the scope of the present disclosure is not limited to these examples.

### Example 1. Preparation of an HIrb Iridium Complex

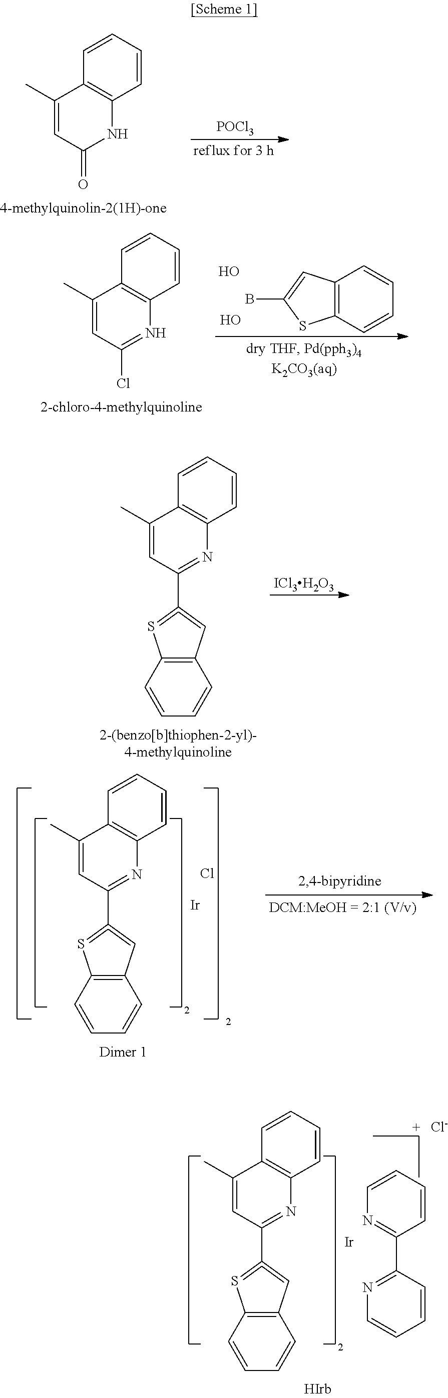

### 2-Chloro-4-methylquinoline

4-Methylquinolin-2(1H)-one (500 mg, 3.14 mmol) was placed in a round-bottom flask, mixed with POCl₃ (5 mL), and refluxed for 3 hours under inert conditions. The reaction mixture was cooled, poured into ice water, and then neutralized using a 2.0 M aqueous NaOH solution. The precipitate was filtered and washed with water. The raw product was crystallized from petroleum ether. ¹HNMR (400 MHz, DMSO-d⁶) δ8.10 (d, J=8.3 Hz, 1H), 7.92 (d, J=8.5 Hz, 1H), 7.80 (t, J=7.4 Hz, 1H), 7.66 (t, J=7.3 Hz, 1H), 7.47 (s, 1H), 2.68 (s, 3H).

### 2-(Benzo[b]thiophen-2-yl)-4-methylquinoline

A mixture of 2-chloro-4-methylquinoline (300 mg, 1.68 mmol), benzo[b]thiophene-2-boronic acid (360.8 mg, 2.03 mmol), and tetrakis(triphenylphosphine) palladium(0) (97.1 mg, 0.084 mmol) was dissolved in a dry solvent (toluene/ethanol=3:1 (volume ratio)) under a nitrogen gas atmosphere, and then a degassed K₂CO₃-containing aqueous solution (2.8 M, 2 mL) was injected. The reaction mixture was reacted overnight under reflux conditions and then neutralized with H₂O. The raw product was extracted with ethyl acetate and purified by column chromatography with silica gel (solvent conditions: hexane/ethyl acetate=50:1). 1H NMR (400 MHz, DMSO-d6) δ 8.34 (s, 1H), 8.17 (s, 1H), 8.09 (d, J=7.1 Hz, 1H), 8.05-7.95 (m, 2H), 7.90 (dt, J=7.3, 3.6 Hz, 1H), 7.77 (t, J=6.9 Hz, 1H), 7.61 (t, J=7.6 Hz, 1H), 7.45-7.36 (m, 2H), 2.75 (s, 3H).

### Dimer1

2-(Benzo[b]thiophen-2-yl)-4-methylquinoline (250 mg, 0.9 mmol) and IrCl3·H2O (135.5 mg, 0.45 mmol) were dissolved in 10 mL of a solvent mixture (2-methoxyethanol/water=3:1 by volume ratio) and reacted under reflux conditions for 24 hours. After the reaction, the mixture was neutralized with H₂O and filtered. The filtered red doublets were dried overnight in a vacuum oven to be used in the next step without additional purification.

### HIrb

Dimer1 (50 mg, 0.0322 mmol) and 2,2′-bipyridine (15.1 mg, 0.097 mmol) were dissolved in 10 mL of a solvent mixture (methanol/dichloromethane=1:1 by volume ratio) and reacted overnight under reflux conditions. The solvent was removed under reduced pressure, and the remaining solid material was dissolved in a small amount of ethanol (approximately 5 mL), followed by filtration. The filtrate was concentrated in vacuo and precipitated with 1 mL of dichloromethane and 20 mL of hexane. ¹H NMR (400 MHz, DMSO-d⁶) δ 8.37 (d, J=8.3 Hz, 2H), 8.29 (d, J=5.6 Hz, 2H), 8.08 (d, J=8.7 Hz, 4H), 7.96 (dd, J=17.2, 8.2 Hz, 4H), 7.75-7.71 (m, 2H), 7.33 (t, J=7.5 Hz, 2H), 7.14 (t, J=7.6 Hz, 2H), 6.93 (dt, J=15.6, 8.7 Hz, 4H), 6.65 (t, J=7.7 Hz, 2H), 6.21 (d, J=8.3 Hz, 2H), 2.90 (s, 6H).

An HIrb iridium complex represented by the following Chemical Formula 2 was prepared via the preparation method above.

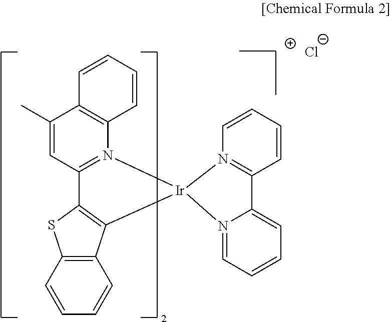

### Example 2. Preparation of FIrb Iridium Complexes

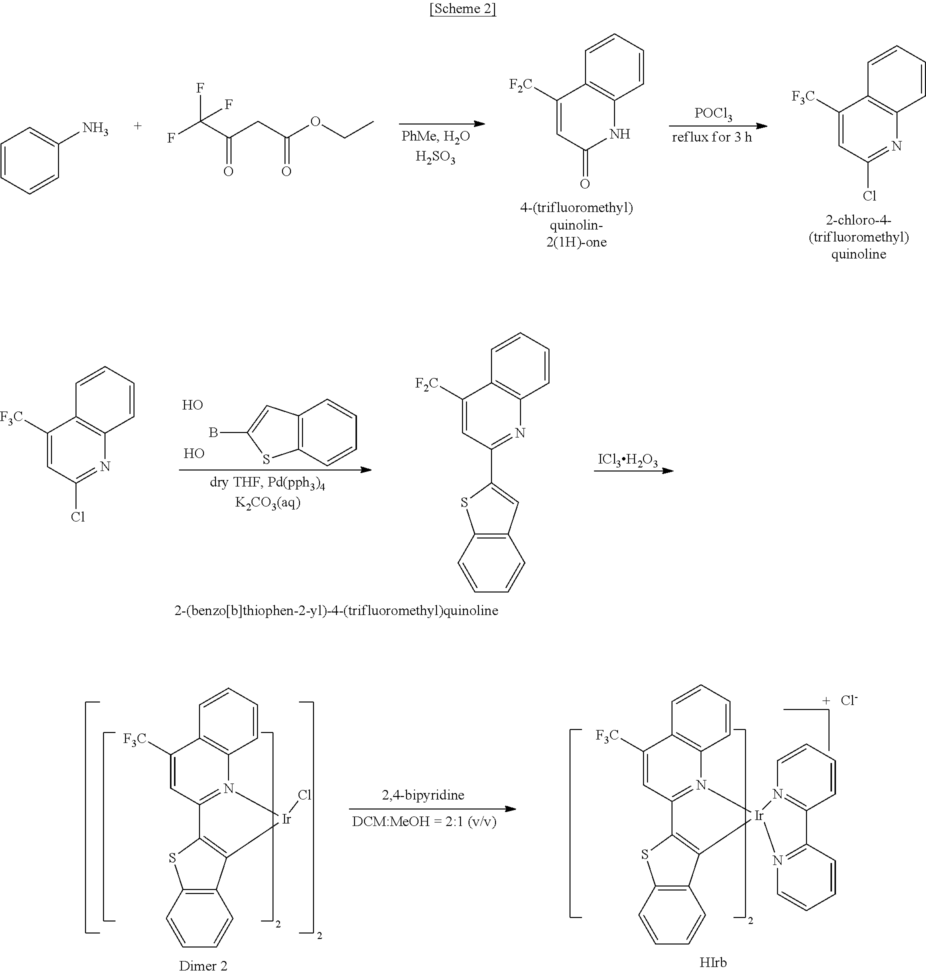

### 4-(Trifluoromethyl)quinolin-2(1H)-one

A mixture of aniline (3.2 g, 33 mmol) and 2-chloro-4-(trifluoromethyl)quinoline (6.2 g, 39 mmol) was dissolved in degassed toluene (100 mL) and refluxed for 10 minutes. After adding a small amount of water (500 μL), the reaction mixture was refluxed overnight, followed by concentration and distillation. The concentrated raw product was injected dropwise into heated H₂SO₄ at 80° C. and stirred at the same temperature for 1 hour. After cooling, the reaction mixture was poured into a beaker containing ice. The resulting solid product was filtered and washed with dry ether. ¹H NMR (400 MHz, DMSO-d⁶) δ 12.33 (s, 1H), 7.69 (d, J=8.5 Hz, 1H), 7.63 (t, J=7.8 Hz, 1H), 7.43 (d, J=8.3 Hz, 1H), 7.30 (t, J=7.7 Hz, 1H), 6.97 (s, 1H).

### 2-Chloro-4-(trifluoromethyl)quinoline

4-(Trifluoromethyl)quinolin-2(1H)-one (1 g, 4.69 mmol) was placed in a round-bottom flask, mixed with POCl₃ (10 mL), and refluxed for 3 hours under inert conditions. The reaction mixture was cooled, poured into ice water, and then neutralized using a 2.0 M aqueous NaOH solution. The precipitate was filtered and washed with water. The raw product was crystallized from petroleum ether. ¹H NMR (400 MHz, DMSO-d⁶) δ 8.22-8.02 (m, 3H), 7.99 (t, J=7.7 Hz, 1H), 7.86 (t, J=7.7 Hz, 1H).

### 2-(Benzo[b]thiophen-2-yl)-4-(trifluoromethyl)quinoline

A mixture of 2-chloro-4-(trifluoromethyl)quinoline (220 mg, 0.95 mmol), benzo[b]thiophene-2-boronic acid (203 mg, 1.14 mmol), and tetrakis(triphenylphosphine) palladium(0) (54.9 mg, 0.048 mmol) was dissolved in a dry solvent (toluene/ethanol=3:1 (volume ratio)) under a nitrogen gas atmosphere, and then a degassed K₂CO₃-containing aqueous solution (2.8 M, 2 mL) was injected. The reaction mixture was reacted overnight under reflux conditions and then neutralized with H₂O. ¹H NMR (400 MHz, DMSO-d6) δ 8.64 (d, J=2.9 Hz, 2H), 8.18 (d, J=8.4 Hz, 1H), 8.08 (d, J=8.6 Hz, 1H), 8.07-7.98 (m, 1H), 7.98-7.85 (m, 2H), 7.80 (t, 1H), 7.50-7.36 (m, 2H).

### Dimer2

2-(Benzo[b]thiophen-2-yl)-4-(trifluoromethyl)quinoline (175 mg, 0.532 mmol) and IrCl₃·H₂O (79.3 mg, 0.266 mmol) were dissolved in 10 mL of a solvent mixture (2-methoxyethanol/water=3:1 by volume ratio) and reacted under reflux conditions for 24 hours. After the reaction, the mixture was neutralized with H₂O and filtered. The filtered red doublets were dried overnight in a vacuum oven to be used in the next step without additional purification.

### FIrb

Dimer2 (63 mg, 0.0819 mmol) and 2,2′-bipyridine (39 mg, 0.2496 mmol) were dissolved in 12 mL of a solvent mixture (methanol/dichloromethane=1:1 by volume ratio) and reacted overnight under reflux conditions. The solvent was removed under reduced pressure, and the remaining solid material was dissolved in a small amount of ethanol (approximately 5 mL) followed by filtration. The filtrate was concentrated in vacuo and precipitated with 1 mL of dichloromethane and 20 mL of hexane. ¹H NMR (400 MHz, DMSO-d⁶) δ 8.67 (d, J=4.9 Hz, 1H), 8.43 (s, 1H), 8.38 (d, J=7.9 Hz, 1H), 8.34 (d, J=8.2 Hz, 1H), 8.25 (d, J=5.6 Hz, 1H), 8.15-8.04 (m, 2H), 7.95 (d, J=7.8 Hz, 1H), 7.91 (d, J=8.6 Hz, 1H), 7.74 (t, J=6.7 Hz, 1H), 7.49 (t, J=7.6 Hz, 1H), 7.44 (d, J=6.2 Hz, 1H), 7.24 (t, J=7.6 Hz, 1H), 7.08 (dt, J=15.8, 8.7 Hz, 2H), 6.77 (t, J=7.6 Hz, 1H), 6.37 (d, J=8.3 Hz, 1H).

An FIrb iridium complex represented by the following Chemical Formula 3 was prepared through the preparation method above.

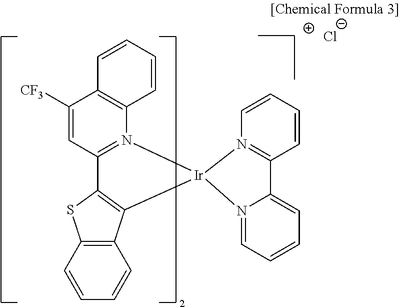

### Comparative Example 1. Preparation of an HIra Iridium Complex

### HIra

Dimer1 (50 mg, 0.032 mmol) and sodium carbonate (34.13 mg, 0.322 mmol) were dissolved in 10 mL of 1,2-dichloroethane solvent, and acetyl acetone (16.1 mg, 0.161 mmol) was added and reacted under reflux conditions for 12 hours. The solvent was removed under reduced pressure, and the remaining material was purified by silica gel column chromatography (eluent: DCM:Hex=2:1 v/v). ¹H NMR (400 MHz, DMSO-d⁶) δ 8.06-8.02 (m, 2H), 7.94-7.89 (m, 4H), 7.84-7.76 (m, 2H), 7.48-7.43 (m, 2H), 7.38 (t, J=7.7 Hz, 2H), 7.04-6.98 (m, 2H), 6.54 (dd, J=8.3, 7.1 Hz, 2H), 6.15 (d, J=8.2 Hz, 2H), 2.98-2.96 (m, 6H), 1.47 (s, 6H).

An HIra iridium complex represented by the following Chemical Formula 4 was prepared through the preparation method above.

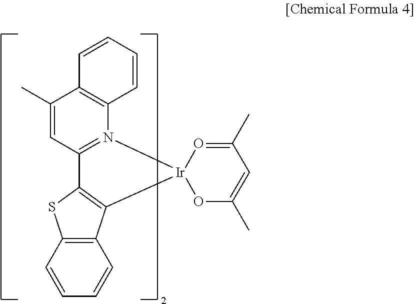

### Comparative Example 2. Preparation of an FIra Iridium Complexes

### FIra

Dimer2 (50 mg, 0.028 mmol) and sodium carbonate (29.68 mg, 0.28 mmol) were dissolved in 10 mL of 1,2-dichloroethane solvent, and acetyl acetone (14 mg, 0.14 mmol) was added and reacted under reflux conditions for 12 hours. The solvent was removed under reduced pressure, and the remaining material was purified by silica gel column chromatography (eluent: DCM:Hex=3:1 v/v). ¹H NMR (400 MHz, DMSO-d⁶) δ 8.33 (s, 2H), 8.03 (s, 2H), 7.89 (dd, J 12.5, 8.4 Hz, 5H), 7.52 (dt, J=27.9, 8.0 Hz, 4H), 7.10 (d, J=7.8 Hz, 2H), 6.66 (t, J=7.6 Hz, 3H), 6.20 (d, J=8.2 Hz, 2H), 4.73 (s, 1H), 1.21 (s, 6H).

An FIra iridium complex represented by the following Chemical Formula 5 was prepared through the preparation method above.

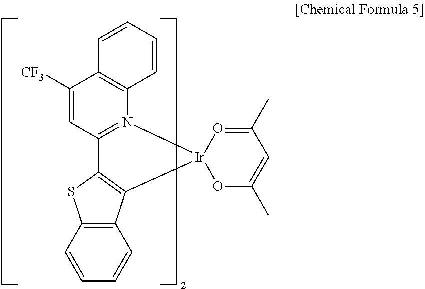

### Comparative Example 3. Preparation of an FIrp Iridium Complex

### FIrp

Dimer2 (450 mg, 0.254 mmol), hydroxyl picolinic acid (106.14 mg, 0.763 mmol), and sodium carbonate (269.61 mg, 2.544 mmol) were added and dissolved in 30 mL of ethoxy ethanol. Reflux was performed overnight to give rise to a reaction. The reactant was extracted with ethyl acetate and purified through column chromatography (eluent: DCM).

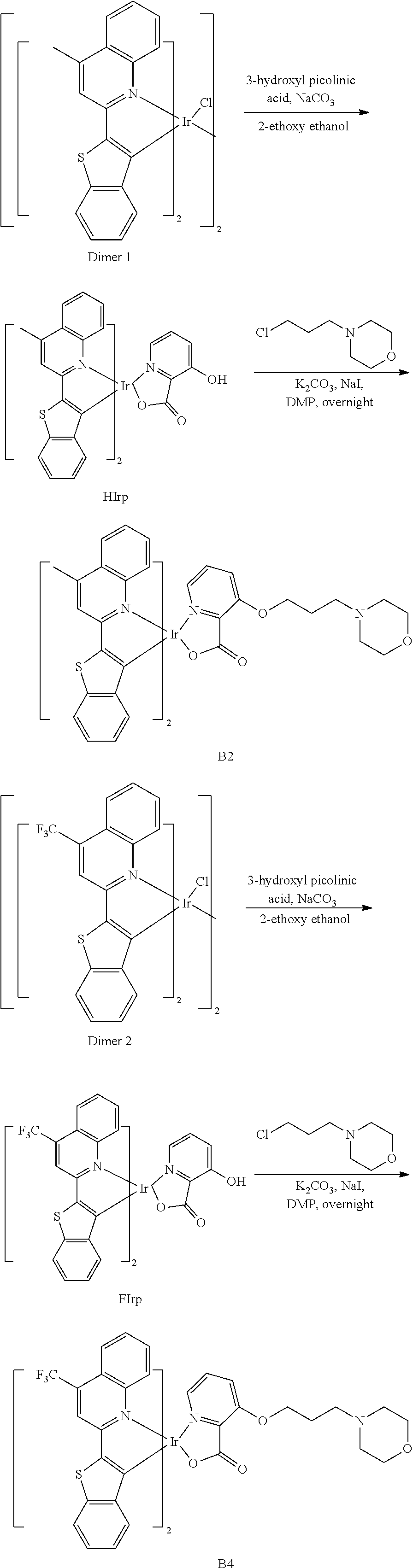

An FIrp iridium complex represented by the following Chemical Formula 6 was prepared through the preparation method above.

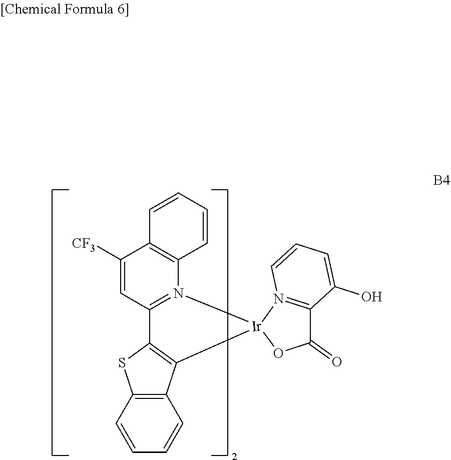

In addition, a mixture of 1 equivalent of FIrp and 2.2 equivalents of K₂CO₃ was dissolved in degassed dry DMF under a nitrogen gas atmosphere and then stirred at room temperature for 30 minutes. 1.5 equivalents of sodium iodide dissolved in DMF and 1.5 equivalents of 4-(3-chloropropyl)morpholine were injected into the reaction mixture, which was then refluxed overnight. The raw product was extracted with ethyl acetate and purified by column chromatography (solvent conditions: dichloromethane/methanol=10:1 by volume ratio).

A B4 iridium complex represented by the following Chemical Formula 9 was prepared through the preparation method above.

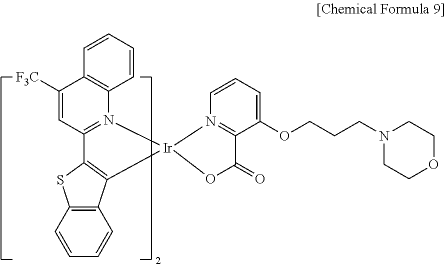

### Comparative Example 4. Preparation of a B2 Iridium Complex

### HIrp

1 equivalent of Dimer1, 3 equivalents of hydroxyl picolinic acid, and 10 equivalents of sodium carbonate were added and dissolved in 30 mL of ethoxy ethanol. Reflux was performed overnight to give rise to a reaction. The reactant was extracted with ethyl acetate and purified through column chromatography (eluent: DCM).

### B2

A mixture of 1 equivalent of HIrp and 2.2 equivalents of K₂CO₃ was dissolved in degassed dry DMF under a nitrogen gas atmosphere and stirred at room temperature for 30 minutes. 1.5 equivalents of sodium iodide dissolved in DMF and 1.5 equivalents of 4-(3-chloropropyl)morpholine were injected into the reaction mixture, which was then refluxed overnight. The raw product was extracted with ethyl acetate and purified by column chromatography (solvent conditions: dichloromethane/methanol=10:1 by volume ratio).

A B2 iridium complex represented by the following Chemical Formula 7 was prepared through the preparation method above.

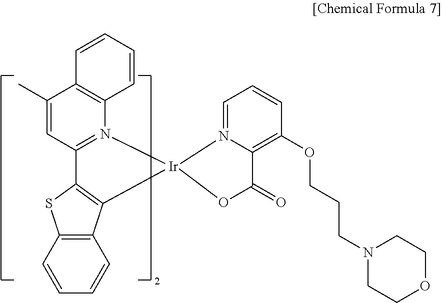

### Comparative Example 5. Preparation of a TIr3 Iridium Complex

The chlorine-linked iridium dimer (intermediate complex) was prepared using 2 equivalents of IrCl₃·nH₂O (Strem Chemicals, USA) and 4 equivalents of C\hat{ }N ligand (C\hat{ }N Ligand 2pq: Sigma Aldrich, USA). One equivalent of the chlorine-linked iridium dimer prepared above and two equivalents of 2,2′-bipyridine (Sigma Aldrich, USA) were dissolved in a mixed solvent of dichloromethane (DCM: Samchun Chemicals, Korea) and methanol (MeOH: Samchun Chemicals, Korea) (DCM:MeOH=1:1 by volume ratio), and the mixture was refluxed and reacted for 12 hours in the presence of inert gas N2. Afterwards, precipitation was followed using DCM and hexane (n-hexane: Samchun Chemicals, Korea), and a TIr3 iridium complex represented by Chemical Formula 8 was prepared through an additional washing process using hexane.

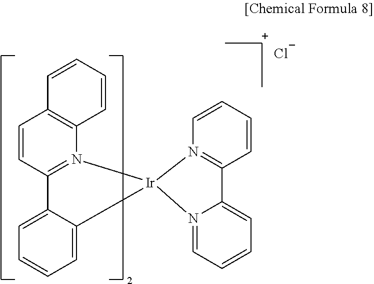

### Experimental Example 1. Comparative Experiment for an Intracellular Accumulation Rate Depending on the Charges of the Iridium Complex

HeLa cells were seeded on 60 π cell culture dishes and cultured until the cells grew to about 70-80% confluent. These cells were treated with DMEM containing 10 μM of each iridium complex, which was carried out for 2 hours. Afterwards, the cells were rinsed three times with warm phosphate-buffered saline (PBS) and detached using trypsin. The number of separated cells (1 mL) was counted followed by transfer to a glass vessel, and then 1 mL of 65% HNO₃ (TraceMetal™ Grade, Thermo Fisher) was added to the cell solution and heated at 90° C. for 1 hour. The digested solution was diluted with ultrapure water (4 mL), and then the amount of iridium element was analyzed using inductively coupled plasma optical emission spectroscopy (ICP-OES) (700-ES, Varian Inc.).

The intracellular accumulation rate was compared by calculating the concentration of iridium element contained per cell based on the results.

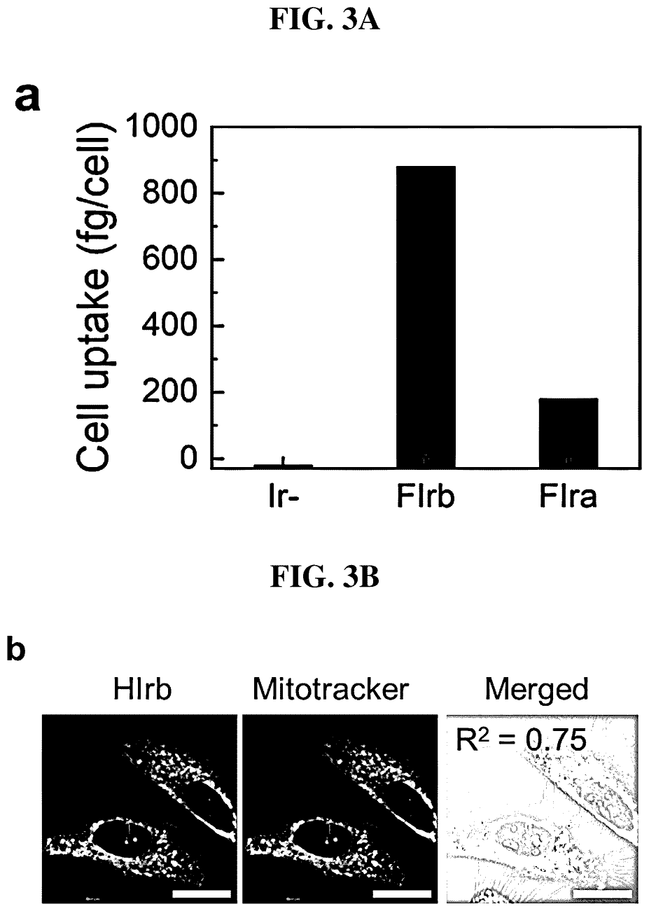

FIG. 3A shows a graph illustrating changes in an intracellular accumulation rate depending on the charge of iridium complexes in Example 2 (FIrb) and Comparative Example 2 (FIra) obtained using inductively coupled plasma optical emission spectroscopy (ICP-OES), and FIG. 3B shows cell images regarding the merging of intracellular signals (green) and mitotracker signals (red) of the iridium complex of Example 1 (HIrb), obtained using a confocal microscope.

Due to the negatively charged phospholipid layer of the cell membrane, it is possible to increase the intracellular accumulation rate when the drugs is positive charged. The iridium complex of the present disclosure secures a higher intracellular accumulation rate than an uncharged iridium complex using acetyl acetone or picolinic acid as a sub-ligand, through a method of forming the positive charge on the iridium complex by adopting bipyridine as a sub-ligand.

Specifically, due to the strong negative voltage inside the mitochondria, lipid-soluble cations easily pass through the plasma and mitochondrial membranes to be directly taken up into the mitochondria. Accordingly, it was found that the positively charged iridium complex of the present disclosure was accumulated in large quantities in mitochondria due to electrostatic attraction (FIG. 3B).

### Experimental Example 2. Comparative Experiment for a Reactive Oxygen Generation Ability

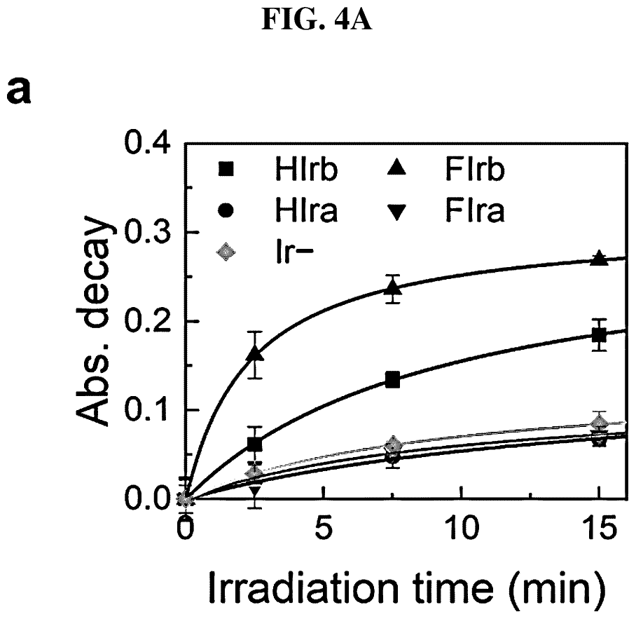

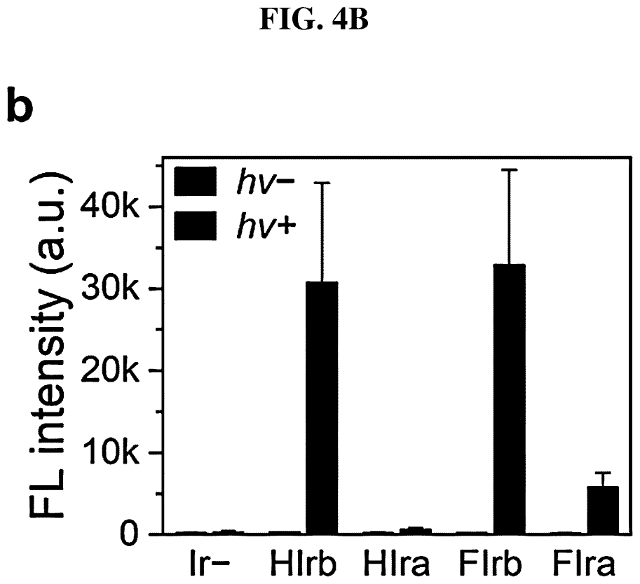

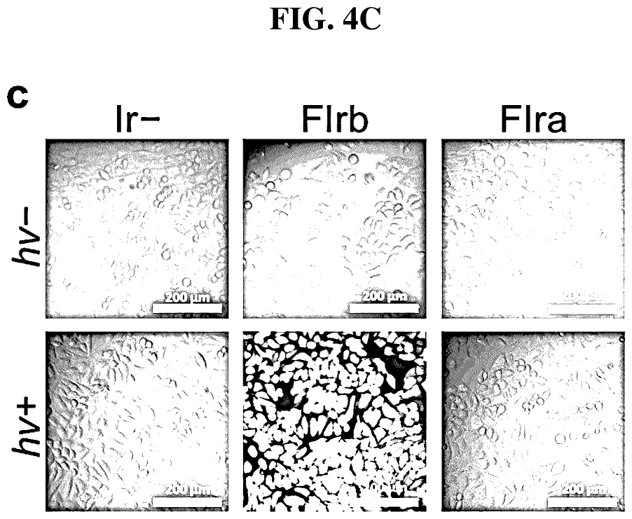

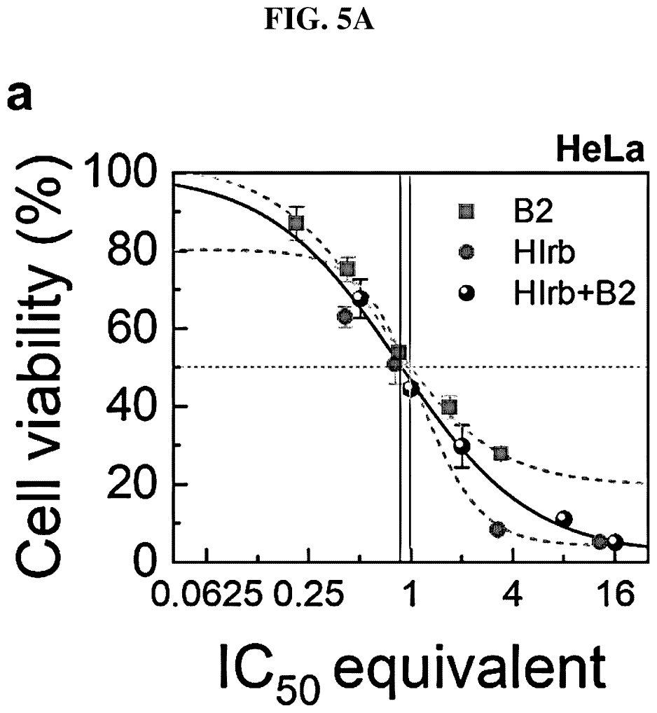

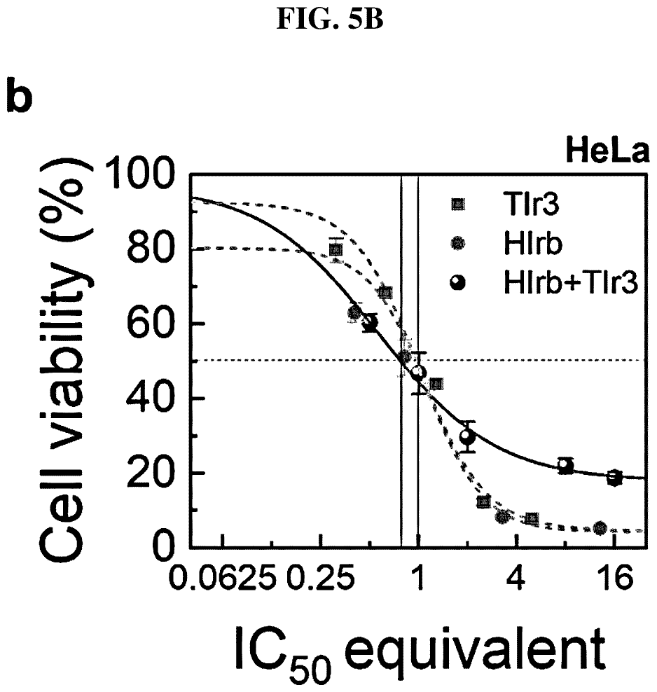

FIG. 4A shows a graph of changes in absorption of 9,10-anthracenediyl-bis(methylene)dimalonic acid (ABDA) according to an amount of singlet oxygen (¹O₂) produced by light irradiation time (0.25 sun, 25 mW/cm²). A 100 mM high-dosage ABDA solution was added to a 4 μM iridium complex solution to create a sample with a final concentration of 100 μM ABDA and 4 μM iridium complex. The prepared samples were irradiated with 1 sun, 25 mW/cm² for 0, 2.5, 7.5, and 15 minutes, and the absorbance of ABDA at 384 nm was measured using a microplate reader. FIG. 4B shows a graph illustrating a fluorescence intensity (light conditions: 1 sun, 100 mW/cm², 60 s) which increases in response to type I reactive oxygen of Dihydrorhodamine 123 (DHR123) which is a fluorescent substance. A high-dosage solution of DHR 123 (4 mM) was mixed with a 4 μM iridium complex solution in a volume ratio of 1000:1 to obtain a sample with a final concentration of 4 μM TIr3 and DHR 123. Each sample was irradiated with 1 sun, 100 mW/cm² for 0 and 10 minutes, and the green fluorescence from rhodamine 123 was recorded for each sample using a microplate reader (λ (absorption)=507 nm and λ (emission)=529 nm). FIG. 4C shows images illustrating the intracellular fluorescence analysis of 2′,7′-dichlorodihydrofluorescein diacetate (DCFH-DA, an intracellular type I reactive oxygen marker) depending on the presence of light irradiation (0.25 sun, 25 mW/cm², 1 min). 2′,7′-Dichloro-dihydrofluorescein diacetate (H2DCF-DA) is a ROS indicator that acts within living cells. After culture on confocal microscopy dishes until a cell confluency reached 60%, cells were treated with 4 μM iridium complex for 2 hours. After washing several times with DPBS, HeLa cells were treated with DMEM containing 10 μM DCFH-DA for 30 minutes. The cells were then exposed to light emitted by an LED array corresponding to the absorption region of each iridium complex, the fluorescence of the activated DCFH-DA was recorded using an LSM780 multiphoton confocal microscope, and the integral of the fluorescence intensity of the cells was detected using Zen blue software.

Thereby, it may be found that the iridium complexes (HIrb, FIrb) of Examples 1 and 2 of the present disclosure having bipyridine as a sub-ligand exhibit higher reactive oxygen generating abilities than the iridium complexes (HIra, FIra, FIrp) in Comparative Examples 1 to 3.

### Experimental Example 3. Comparative Experiment for Cancer Cell Killing Ability

After seeding 1.5×10⁴ cells in a 96-well plate, the cells were cultured overnight at 37° C. under 5% carbon dioxide conditions. Afterwards, the cells were treated with the iridium complex and cultured for 2 hours, after which the medium was replaced with fresh medium. After irradiated for 30 seconds with a 1 sun simulator (100 mW/cm2, PEG-L01, Peccell), cells were cultured for an additional 24 hours in a humidified incubator containing 5% carbon dioxide at 37° C. Cell viability was measured using the MTT (tetrazolium salt (3-(4,5-dimethylthiazol-2-yl)-2,5-diphenyltetrazolium bromide)) assay. MTT reagent was added to each well and cultured for 4 hours, and then the formazan crystals produced in the surviving cells were dissolved in DMSO, followed by measurement of the absorbance at 570 nm.

Through these experiments, the IC₅₀ values (light conditions: 1 sun, 3 J/cm²) of the iridium complexes of Examples 1 and 2 and Comparative Examples 1 to 3 of the present disclosure under the same light conditions are as shown in Table 1 below. Here, the IC₅₀ value refers to a concentration at which 50% of cancer cells are killed when photodynamic therapy is carried out on cancer cells with a single drug.

TABLE 1

| | | | Comparative | Comparative | Comparative |
|---|---|---|---|---|---|
| | Example 1 | Example 2 | Example 1 | Example 2 | Example 3 |
| | HIrb | FIrb | HIra | FIra | FIrp |
| A549 | 0.16 ± 0.00 | 0.98 ± 0.05 | >32 | >32 | >32 |
| HeLa | 0.22 ± 0.02 | 0.85 ± 0.23 | >32 | >32 | >32 |
| PANC-1 | 0.99 ± 0.07 | 0.53 ± 0.04 | >32 | >32 | >32 |

The iridium complexes of Examples 1 and 2, which have a high accumulation rate in cancer cells as well as excellent ability to generate reactive oxygen, showed a striking difference in cancer cell killing efficiency at the same light intensity and the same drug concentration compared to the iridium complexes of Comparative Examples 1 to 3.

In the case of the iridium complexes of Comparative Examples 1 to 3, 100% cell viability was observed even at a concentration of 32 μM (cancer cell killing efficiency was close to 0), whereas in the case of the iridium complex of Example 1 (HIrb), it was found that 50% of cancer cells were killed in HeLa (cervical cancer) even at 0.22±0.02 μM, and in the case of FIrb, the IC₅₀ value was observed at 0.51±0.30 μM.

It was determined that the effect of the present disclosure on cancer cell death showed a consistent tendency not only in HeLa (cervical cancer), but also in A549 (lung cancer) and PANC-1 (pancreatic cancer).

### Experimental Example 4. Experiment for Synergy Effects Between Iridium Complexes

To identify the synergy effect, the effective concentrations of the drugs were compared using the equation presented in a previous study in ‘Frontiers in bioscience (Elite edition), 2010, 2(1), 241-249’ (Equation 1).

$${IC}_{50} Equiv . = \frac{Conc . ( A )}{{IC}_{50} ( A )} + \frac{Conc . ( B )}{{IC}_{50} ( B )} \qquad [ Equation 1 ]$$

When the same light (1 sun, 3 J/cm²) was irradiated to cancer cells administered with the HBrb, B2, and TIr3 iridium complexes as respective photosensitizers, the IC₅₀ values for the respective photo sensitizers were first calculated, and the results are shown in Table 2 below.

TABLE 2

| | hv− | | | hv+ | | |
|---|---|---|---|---|---|---|
| | | Comparative | Comparative | | Comparative | Comparative |
| | Example 1 | Example 4 | Example 5 | Example 1 | Example 4 | Example 5 |
| | HIrb | B2 | TIr3 | HIrb | B2 | TIr3 |
| A549 | 13.35 ± 1.65 | >32 | 19.76 ± 1.99 | 0.16 ± 0.00 | 3.80 ± 1.30 | 3.12 ± 0.20 |
| HeLa | 10.14 ± 1.84 | >32 | 8.77 ± 0.73 | 0.22 ± 0.02 | 2.40 ± 0.08 | 1.53 ± 0.07 |
| MDA-MB-231 | 11.57 ± 0.64 | >32 | 7.51 ± 0.84 | 0.11 ± 0.04 | 0.89 ± 0.10 | 0.89 ± 0.02 |
| PANC-1 | 26.31 ± 2.81 | >32 | 10.69 ± 0.39 | 0.99 ± 0.07 | 11.72 ± 2.02 | 3.86 ± 0.18 |
| Caco-2 | 9.23 ± 0.16 | >32 | 7.88 ± 0.13 | 0.12 ± 0.03 | 0.68 ± 0.01 | 0.89 ± 0.09 |
| MKN45 | 17.54 ± 2.69 | >32 | 16.30 ± 0.17 | 0.40 ± 0.12 | 0.86 ± 0.03 | 0.55 ± 0.06 |

Afterwards, the two drugs whose synergy effect was to be identified were mixed at an IC₅₀ ratio and administered to cancer cells. (Specific experimental method: 1.5×10⁴ cells were seeded in a 96-well plate and cultured overnight at 37° C. in the presence of 5% carbon dioxide. Afterwards, the cells were treated with the iridium complex combination and cultured for 2 hours, after which the medium was replaced with fresh medium. Cells were irradiated for 30 seconds with a 1 sun simulator (100 mW/cm2, PEG-L01, Peccell) and then cultured for an additional 24 hours in a humidified incubator containing 5% carbon dioxide at 37° C. Cell viability was measured using the MTT (tetrazolium salt (3-(4,5-dimethylthiazol-2-yl)-2,5-diphenyltetrazolium bromide)) assay. MTT reagent was added to each well followed by culture for 4 hours, formazan crystals were dissolved in DMSO, and the absorbance was measured at 570 nm.

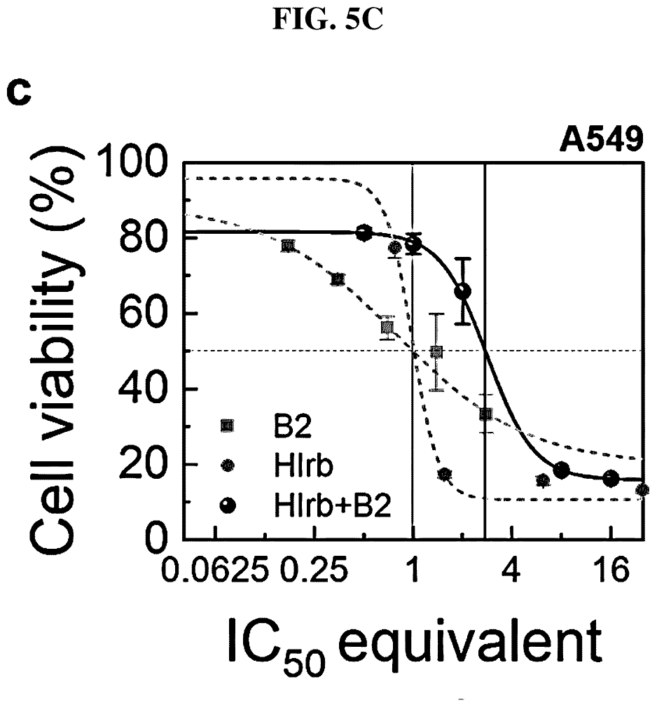

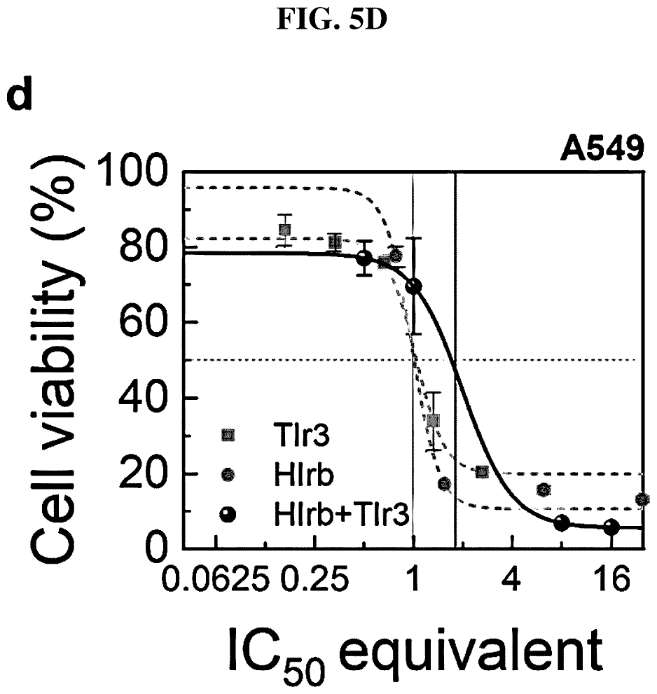

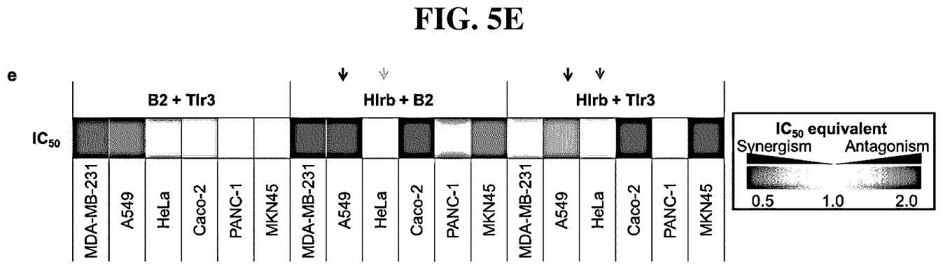

In this process, the drug concentration between iridium complex combinations was expressed as IC₅₀ equivalent, which is a relative value of the IC₅₀ value, rather than a unit of M (Equation 1 and FIGS. 5A-5E). Here, if the IC₅₀ equivalent value of the combination of the two drugs is lower than the condition with single drug (IC₅₀ equivalent=1), it may be interpreted that the combination has a synergism (FIGS. 5A and 5B), and if the IC₅₀ equivalent value is large, it may be expressed as having an antagonism (FIGS. 5C and 5D).

To secure higher cancer cell killing efficiency as described above, the conditions that can induce a synergy effect with the HIrb iridium complex were monitored, and the results of these experiments are shown in FIGS. 5A to 5E. Specifically, FIGS. 5A to 5D show the cell viability according to the IC₅₀ equivalent value calculated by Equation 1, graphed with cases in which a combination of HIrb and B2, a lysosome-targeting iridium complex, was administered to HeLa, a cervical cancer cell line (FIG. 5A), a combination of HIrb and TIr3, a endoplasmic reticulum-targeting iridium complex, was administered (FIG. 5B), a combination of HIrb and B2 was administered to A549, a lung cancer cell line (FIG. 5C), and a combination of HIrb and TIr3 was administered (FIG. 5D). In addition, the degree of synergy of each combination (B2+TIr3, HIrb+B2, HIrb+TIr3) for various cancer cell lines (MDA-MB-231, breast cancer; A549, lung cancer; HeLa, cervical cancer; Caco-2, colon cancer; PANC-1, pancreatic cancer; MKN45, gastric cancer) was schematically plotted in FIG. 5E.

As a result, the iridium complex HIrb, which showed higher photodynamic therapeutic efficacy than the iridium complexes B2 and TIr3 which target lysosomes and endoplasmic reticulum, respectively, showed a synergy effect in the combination of each of B2 and TIr3 in the HeLa cell line of cervical cancer (FIGS. 5A and 5B).

Synergistic drug combinations are of great significance for photosensitizers that are inherently toxic. This is because it means that the amount of toxic photosensitizer required to achieve the same effective photodynamic therapeutic effect may be reduced by more than half. Therefore, as shown in Table 2 above, the fact that toxic HIrb, which showed an IC₅₀, dark of about 10 μM for the HeLa cell line, showed the synergy effect with the non-toxic photosensitizer B2 up to 32 μM may be considered as a discovery of a treatment method that brings reduction in the amount of drug required while retaining the high cancer cell treatment efficacy of HIrb, thereby ultimately lowering the burden on the patient's body.

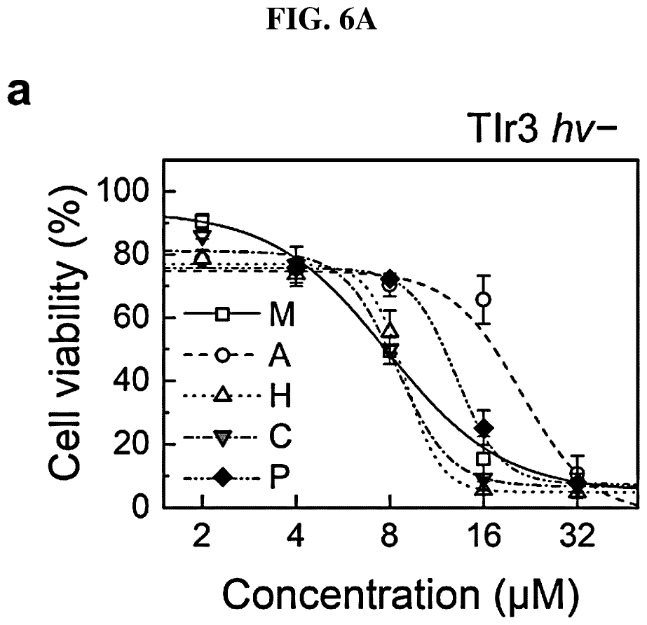

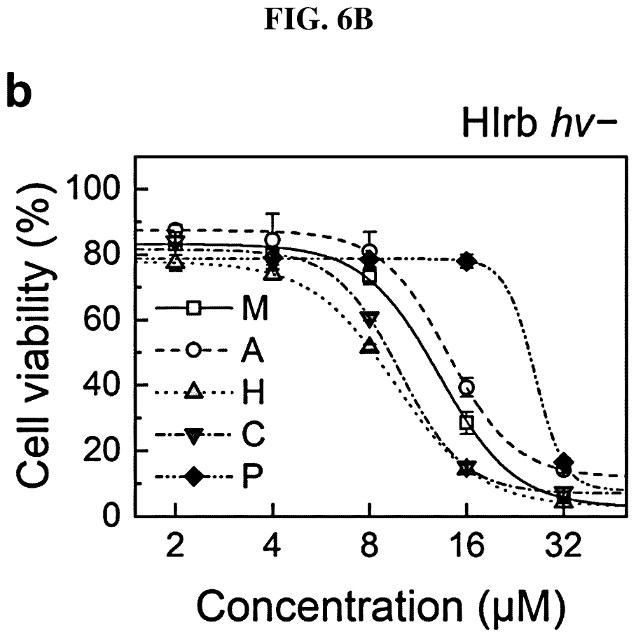

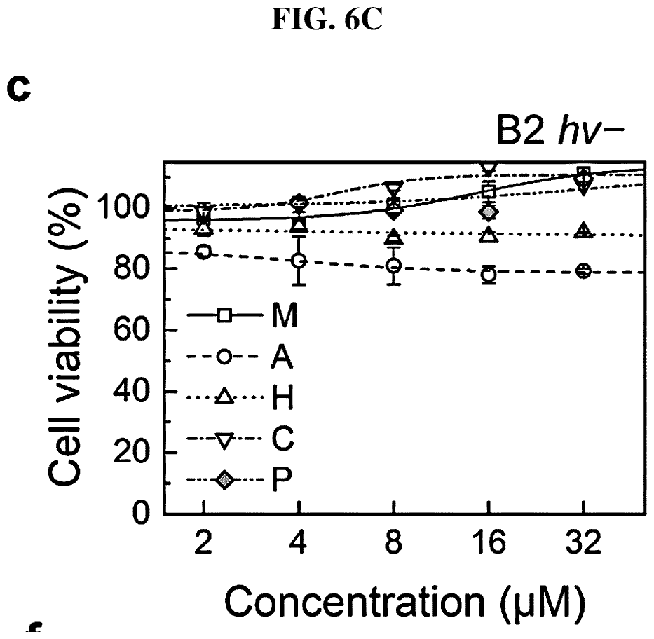

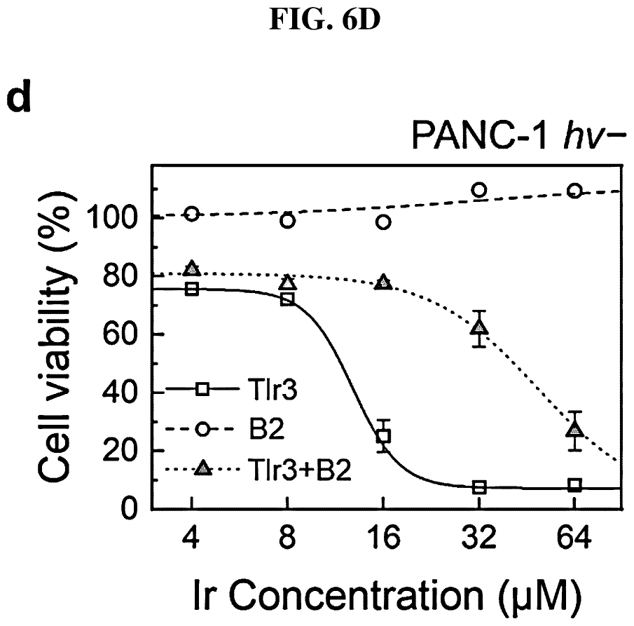

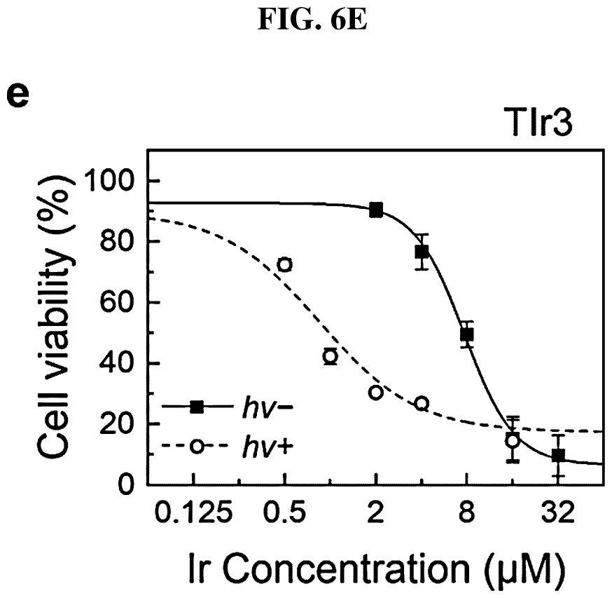

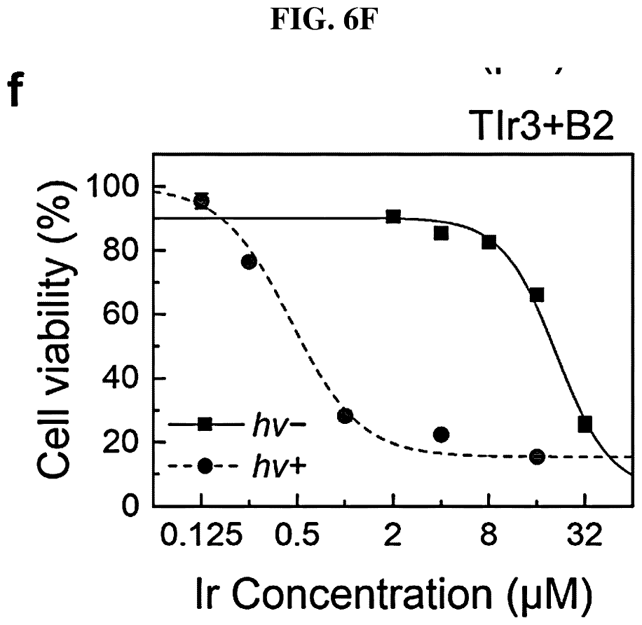

Additionally, the experimental results on the inherent toxicity of the iridium complex are shown in FIGS. 6A-6F. Data indicating the inherent toxicity may be determined by cell viability under conditions without light application (hv−). The experimental results in FIGS. 6A-6F show the cell viability for each cell under conditions where no light was given, where M indicates the cell line MDA-MB-231, A indicates the cell line A549, H indicates the cell line HeLa, C indicates the cell line Caco-2, and P indicates the cell line PANC-1, respectively. FIGS. 6A, 6B, and 6C represent the cell viability when each substance was administered to each cell line for 2 hours, replaced with new media, and then cultured for an additional 24 hours. FIG. 6D is a graph of cell viability when TIr3 and B2 were administered at different concentrations, and at the concentration of a combination of the two (TIr3+B2), to PANC-1, pancreatic cancer cells. In the case of the combination mixed with B2, which has excellent biocompatibility compared to TIr3 which shows low cell viability, it is noticed that the cell viability increased at each concentration (especially, 16 and 32 μM). FIGS. 6E and 6F show the results of identifying the change in the cell viability in the MDA-MB-231 breast cancer cell line under conditions with TIr3 only and the TIr3+B2 combination depending on the presence of light irradiation. By observing significant changes in the TIr3+B2 combination, it is possible to secure the ON-OFF of the light-induced toxicity of the photosensitizer and thus maximize the change in cell viability between normal cells with no light reached and cancer cells with light reached when conducting preclinical experiments in the future.

The description above is intended to be illustrative of the present disclosure, and the embodiments disclosed herein are to describe the technical idea of the present disclosure, rather than limiting, such that those skilled in the art to which the present disclosure pertains will appreciate that various modifications and alterations may be made without departing from the technical idea of the present disclosure. Therefore, the scope of protection of the present disclosure should be construed by the appended claims, and technical matters within a scope equivalent thereto should also be construed as included in the scope of rights of the present disclosure.

An iridium complex according to the present disclosure may be utilized in the field of photodynamic therapy, such as a photosensitizer and a composition for photodynamic therapy.

1. An iridium complex represented by the following Chemical Formula 1: 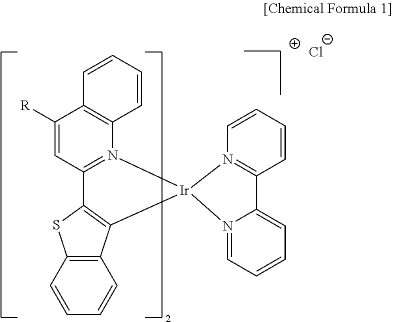
(wherein R is selected from a methyl group (CH₃) or a trifluoromethyl group (CF₃)).

2. A photosensitizer comprising the iridium complex according to claim 1.

3. The photosensitizer of claim 2, wherein the photosensitizer is positively charged.

4. A composition for photodynamic therapy, comprising the photosensitizer according to claim 2 as an active ingredient.

5. A method of treating cancer in a subject, comprising:
administering to the subject the composition of claim 4; and
irradiating light energy to a target tissue of the subject,
wherein the cancer is one or more selected from the group consisting of cervical cancer, colon cancer, pancreatic cancer, lung cancer, breast cancer, stomach cancer, melanoma, skin cancer, bile duct cancer, neuroendocrine tumor, ovarian cancer, liver cancer, bronchial cancer, nasopharyngeal cancer, laryngeal cancer, bladder cancer, colorectal cancer, brain cancer, prostate cancer, bone cancer, head and neck cancer, thyroid cancer, parathyroid cancer, and ureteral cancer.

6. The method of claim 5, wherein the composition further comprises one or more selected from the group consisting of B2, B4, and TIr3.

7. The method of claim 5, wherein the cancer is cervical cancer.

8. The the method of claim 5, wherein reactive oxygen species are generated inside mitochondria by irradiation with light energy.

9. An iridium complex represented by the following Chemical Formula 7 or Chemical Formula 9:

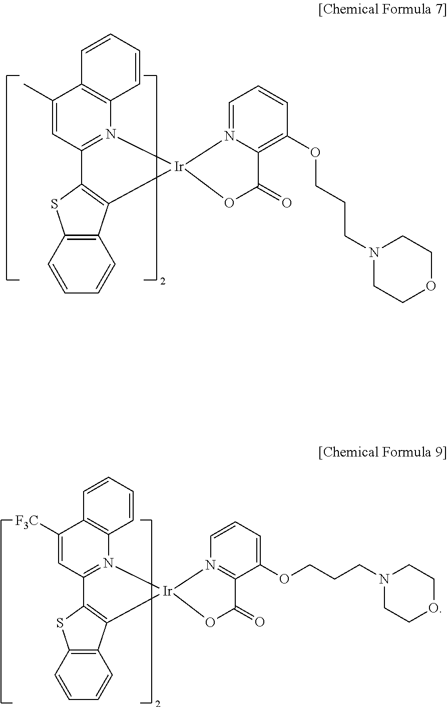

10. A photosensitizer comprising the iridium complex according to claim 9.

11. A composition for photodynamic therapy, comprising the photosensitizer according to claim 10 as an active ingredient.
